SEQUENCE LISTING (3 sequences)

SEQ ID NO: 1 - DNA, 20 bp, synthetic construct
  source 1..20: mol_type = other DNA
agagtttgatcctggctcag

SEQ ID NO: 2 - DNA, 21 bp, synthetic construct
  source 1..21: mol_type = other DNA
tacggctaccttgttacgact

SEQ ID NO: 3 - DNA, 1449 bp, Bacillus velezensis
  source 1..1449: mol_type = genomic DNA
ggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcgg

(19) United States
(12) Patent Application Publication
(10) Pub. No.: US 2023/0337682 A1
(43) Pub. Date: Oct. 26, 2023

(54) BACILLUS VELEZENSIS, FERMENTATION METHOD, FERMENTATION PRODUCT AND ITS APPLICATION

(71) Applicant: Hunan Vegetable Research Institute, Nanchang (CN)

(72) Inventors: Xin Li, Changsha City (CN); Yu Tao, Changsha City (CN); Jie Chen, Changsha City (CN); Fangjun Tan, Changsha City (CN); Chi Zhou, Changsha City (CN); Xuexiao Zou, Changsha City (CN)

(21) Appl. No.: 18/163,866

(22) Filed: Feb. 2, 2023

(30) Foreign Application Priority Data
Apr. 21, 2022 (CN) 202210422749.X

(51) Int. Cl. A01N 63/22; C12N 1/20

(52) U.S. Cl. CPC A01N 63/22; C12N 1/205; C12R 2001/07

(57) ABSTRACT

The disclosure relates to the field of microbial technology, in particular to a strain of Bacillus velezensis XY40-1, a fermentation method, a fermentation product and its application. The Bacillus velezensis XY40-1 was deposited in the China Center for Type Culture Collection on Mar. 29, 2022, with the deposited number of CCTCC NO: M 2022342, and was isolated from the leaves of pepper in Xiangyan Pepper Base, Changsha City, Hunan Province. The Bacillus velezensis XY40-1 and its fermentation metabolite disclosed by the disclosure have good antagonism against Geotrichum candidum, Neopestalotiopsis formicarum and Curvularia.

### CROSS-REFERENCE TO RELATED APPLICATIONS

This application claims the benefit and priority of Chinese Patent Application Number 202210422749.X, filed on Apr. 21, 2022, the disclosures of which are incorporated herein by reference in their entireties.

### Deposited Material

Bacillus velezensis XY40-1, is deposited in “China Center for Type Culture Collection” which has address as “ Wuhan University Wuhan 430072 China” in Mar. 29, 2022, with a CCTCC designation number CCTCC M 2022342. The viability of the culture was tested on Apr. 5 2022, and the deposited culture was viable.

### TECHNICAL FIELD

The present disclosure relates to the field of microbial technology, in particular to a strain of Bacillus velezensis XY40-1, a fermentation method, a fermentation product and its application.

### BACKGROUND

Plant diseases have always been an important factor threatening agricultural production. The massive and long-term use of chemical pesticides not only cause pathogens to develop resistance, but also pesticide residues are harmful to humans, animals and the environment. Biological control is a method that uses beneficial organisms or other organisms to inhibit or eliminate harmful organisms, which has the advantages of non-toxic, harmless, pollution-free and is not easy to produce drug resistance. Therefore, biological control may replace chemical agents as an environmentally safe option in the future.

Endophytic bacteria are isolated from plant tissues that are disinfected on the surface or obtained from the inside of plants, but their presence does not change the phenotypic characteristics and functions of plants. Endophytic bacteria generally exist in higher plants, and endophytic bacteria are diverse in different kinds of plant individuals and different parts. In general, there are more endophytic bacteria in leaves than in other parts, and with the growth of leaves, endophytic bacteria are also increasing. Endophytic bacteria have a stable living space in plants, are not easily affected by environmental conditions, and have an inhibitory effect on some pathogens, and promote the growth of plants. Therefore, endophytic bacteria have potential to become microbial pesticides and yield-increasing bacteria in biological control or be used as potential biocontrol carrier bacteria.

The Chinese application (CN112899196A) has disclosed the application of the Bacillus velezensis in the prevention and treatment of cruciferae clubroot disease; the Chinese application (CN110699275A) has disclosed the antagonism of Bacillus velezensis against botrytis cinerea in postharvest fruits and vegetables; the Chinese application (CN111534460A) has disclosed the efficient antagonism of Bacillus velezensis to Fusarium graminearum.

However, in the existing technology, it has not been studied whether Bacillus velezensis and its fermentation metabolites can antagonize with Geotrichum candidum, Neopestalotiopsis formicarum and Curvularia.

### SUMMARY

In order to solve the above technical problems, the disclosure provides a strain of Bacillus velezensis XY40-1 and its application.

The Bacillus velezensis XY40-1 was stored in China Center for Type Culture Collection on Mar. 29, 2022 at Wuhan University, Wuhan, China, with the deposit number of CCTCC NO: M 2022342.

The microbial inoculum includes the Bacillus velezensis XY40-1.

Preferably, the microbial inoculum is a liquid microbial inoculum or a solid microbial inoculum.

An application of the Bacillus velezensis XY40-1 or the microbial inoculum containing Bacillus velezensis XY40-1 in inhibiting the growth of plant pathogens.

Preferably, the plant pathogen includes any one of Geotrichum candidum, Curvularia and Neopestalotiopsis formicarum.

A fermentation method of Bacillus velezensis XY40-1, which includes the following steps:
(1) preparing of fermentation medium: the formula of the fermentation medium is 0.700% glucose, 1.000% soybean meal, 0.500% magnesium sulfate heptahydrate and 0.014% zinc sulfate heptahydrate; pH is 6.5-7.5;
(2) inoculating Bacillus velezensis XY40-1 with the inoculation amount of 0.7-1.3% into the fermentation medium for fermentation; the fermentation temperature is 30-39° C.; the fermentation speed is 200-240 rpm; the fermentation time is 22-28 hours.

Preferably, in step (1), the pH is 7.3 and the fermentation temperature is 37° C.; in step (2), the inoculation amount is 1.1%, the fermentation temperature is 37° C., the fermentation speed is 220 rpm, and the fermentation time is 24 hours.

The application of the fermentation product obtained by Bacillus velezensis XY40-1 in inhibiting the growth of plant pathogens.

Preferably, the plant pathogen includes any one of Geotrichum candidum, Curvularia and Neopestalotiopsis formarum.

Preferably, the fermentation product contains nogalamycin, rifaximin, selamectin, baicalin and ansamitocin P-3.

The advantageous effects of the present disclosure:

By providing a strain of Bacillus velezensis XY40-1 and its fermentation method, the disclosure finds that Bacillus velezensis XY40-1 and its fermentation metabolite have good antagonism to Geotrichum candidum, Neopestalotiopsis formicarum and Curvularia, so as to provide a new direction for controlling plant diseases.

### BRIEF DESCRIPTION OF THE DRAWINGS

FIG. 1 is the observation photo of Bacillus velezensis XY40-1 under microscope (1000×).

FIG. 2 is the colony morphology of Bacillus velezensis XY40-1 in the front of the plate.

FIG. 3 is the evolutionary tree analysis of Bacillus velezensis XY40-1.

FIG. 4 is the antagonistic effect picture of Bacillus velezensis XY40-1 against Phytophthora capsici, wherein A: Phytophthora capsici; B: effect picture of confrontation culture between Bacillus velezensis XY40-1 and Phytophthora capsici.

FIG. 5 is the antagonistic effect of Bacillus velezensis XY40-1 against pathogens, wherein A: Curvularia; B: effect picture of confrontation culture between Bacillus velezensis XY40-1 and Curvularia.

FIG. 6 is the antagonistic effect of Bacillus velezensis XY40-1 against pathogens, wherein A: Neopestalotiopsis formicarum; B: effect picture of confrontation culture between Bacillus velezensis XY40-1 and Neopestalotiopsis formicarum.

### DETAILED DESCRIPTION OF THE EMBODIMENTS

In order to make the object, technical solution and advantages of the disclosure clearer, the technical solution in the embodiments of the disclosure will be described clearly and completely in combination with the embodiments of the disclosure. It is apparent that the described embodiments are part of the embodiments of the disclosure, not all of them. Based on the embodiments of the disclosure, all other embodiments obtained by the ordinary skilled person in the art without creative work belong to the scope of the disclosure.

The disclosure provides Bacillus velezensis XY40-1, which is isolated from the leaves of capsicum in Xiangyan Pepper Base in Changsha, Hunan Province. The Bacillus velezensis XY40-1 has been stored in the China Center for Type Culture Collection on Mar. 29, 2022, with the deposit number of CCTCC NO: M 2022342. Other microorganisms are all existing technologies.

Embodiment 1: Isolation, Identification, Purification and Preservation of Bacillus velezensis XY40-1

The strain culture medium used in Embodiment 1 is as follows:

LB culture medium (g/L): providing tryptone 10 g/L, yeast extract 5 g/L, sodium chloride 10 g/L, agar powder 15 g/L, dissolving them with water, finally performing constant volume to 1000 mL, pH 7.0±0.2, and autoclaving under 121° C. for 30 min.

Potato Dextrose Agar (PDA) solid medium (g/L): providing 6 g of potato powder, 20 g of glucose, 20 g of agar, dissolving them with water, finally performing constant volume to 1000 mL, pH 5.6±0.2, and autoclaving under 121° C. for 30 min.

(1) Isolation and Identification

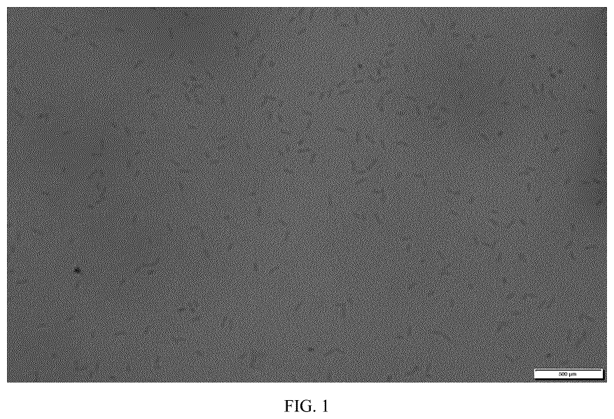

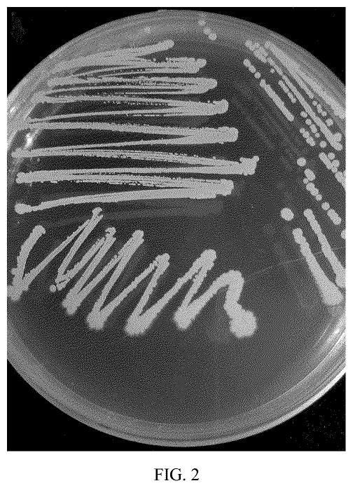

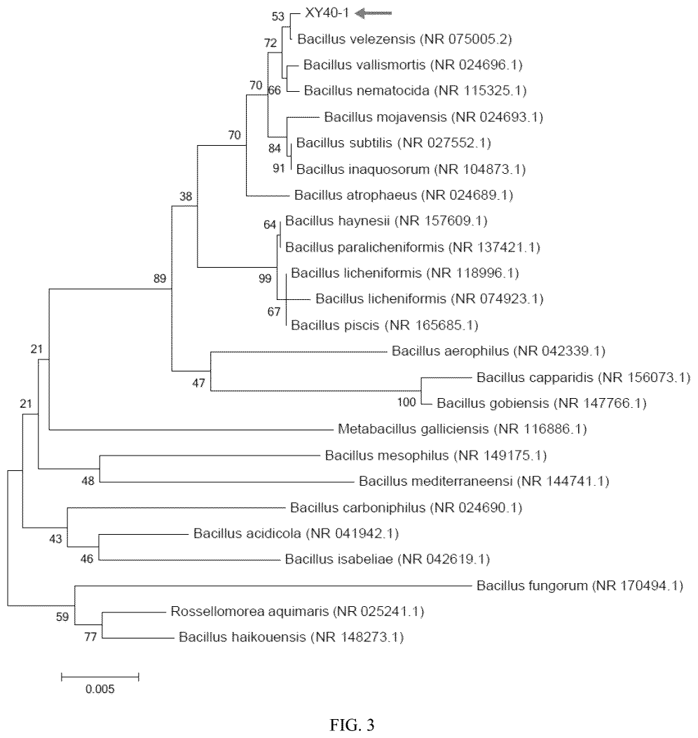

The method for isolation and identification of Bacillus velezensis XY40-1 of the disclosure is as follows: the pepper leaves are taken from Xiangyan Pepper Base in Changsha, the leaves of the pepper on the surface are disinfected, the endophytic bacteria from the leaves are isolated by tissue culture method and spread plate method respectively, and the strains that can inhibit the growth of Phytophthora capsici are screened through plate confrontation experiment. FIG. 1 is the observation photo of Bacillus velezensis XY40-1 under microscope (1000×); FIG. 2 is the colony morphology of Bacillus velezensis XY40-1 in the front of the plate. The strain is purified by streaking on LB plate. The 16SrRNA gene is amplified with the bacterial universal primer (universal primer 27F 5′-GAGTTTGATCCTGGCTCAG -3′ (as shown in SEQ ID No.1) and 1492R 5′-TACGGCTTACCTGTTACGACTT -3′ (as shown in SEQ ID No.2). The gene sequence obtained after the PCR product is sequenced (the nucleotide sequence of the 16SrRNA gene is shown in SEQ ID No.3) is compared and analyzed in NCBI to determine the classification status of the strain. The sequencing results are compared with BLAST on the NCBI website to determine the classification status of closely related bacterial strains. The comparison results shows that the strain has 99.79% genetic relationship with Bacillus velezensis, named as Bacillus velezensis XY40-1. MEGA6.0 is used to construct the phylogenetic tree of Bacillus velezensis XY40-1. The results are shown in FIG. 3.

(2) Purification and Preservation of Bacteria

The plate streaking method is adopted: the colonies that can inhibit the growth of Phytophthora pestis are picked out, a line is streaked on the new LB plate, then it is placed in the biochemical incubator at 28° C. to conduct inverted culture for more than 24 hours, the single colony is picked out and number it XY40-1. Bacillus velezensis XY40-1 strain is inoculated in LB liquid medium, it is incubated in a shaking table at 28° C. and 200 rpm for 24 hours, then 1 mL of fermentation liquid of Bacillus velezensis XY40-1 strain and 1 mL of 50% sterile glycerin are taken, gently shaking and mixing them, and then the mixture are stored at −80° C. for a long time.

Embodiment 2: Antagonism Test of Bacillus velezensis XY40-1 and its Fermentation Metabolite Against Phytophthora, Geotrichum candidum, Neopestalotiopsis formicarum and Curvularia

1, Optimization of Fermentation Conditions of Bacillus velezensis XY40-1

1 Test Strain

Bacillus velezensis XY40-1

2 Preparation of Seed Solution

The strain is inoculated to LB solid plate medium for activation, and cultured at 30° C. for 24 hours. A ring is taken from the activation plate and inoculated into the LB liquid medium with a volume of 50 mL (specification: 100 mL triangular flask), shaking for 24 h at 30° C. and 160 rpm in a shaking table for next use.

3 Single Factor Test

The basic conditions for strain culture are as follows: the seed solution is inoculated into LB liquid medium with 0.5% of the inoculation amount, filling 50% (V/V) of the solution, and the solution is incubated on a shaking table at 30° C. and 160 rpm for 24 hours. Each treatment has 3 replicates. The other elements are unchanged, and the carbon source, the nitrogen source and the inorganic salt in the basic medium are replaced with different carbon source, nitrogen source and inorganic salt respectively. The alternative carbon, nitrogen and inorganic salts and their corresponding prices are as follows: molasses 1.1 yuan/kg, corn starch 1.7 yuan/kg, sucrose 1.8 yuan/kg, glucose 2.3 yuan/kg; soybean meal 2 yuan/kg, corn steep liquor powder 2.9 yuan/kg; magnesium sulfate heptahydrate 0.4 yuan/kg, sodium chloride 0.7 yuan/kg, calcium carbonate 1.8 yuan/kg, manganese sulfate 3.2 yuan/kg. The addition amount of replacement carbon source, nitrogen source and inorganic salt is 0.5%, 1% and 1% respectively.

According to the results of the viable count of Bacillus velezensis, the best component of the culture medium is selected from the following 32 groups of media.

TABLE 1
Experimental groups design

| Serial | | | |
|---|---|---|---|
| number | Nitrogen source | Carbon source | Inorganic salt |
| 1 | Molasses | Soybean meal | Magnesium sulfate |
| | | | heptahydrate |
| 2 | Molasses | Corn steep liquor | Sodium chloride |
| | | powder | |
| 3 | Molasses | Soybean meal | Calcium carbonate |
| 4 | Molasses | Corn steep liquor | Manganese sulfate |
| | | powder | |
| 5 | Corn starch | Soybean meal | Sodium chloride |
| 6 | Corn starch | Corn steep liquor | Magnesium sulfate |
| | | powder | heptahydrate |
| 7 | Corn starch | Soybean meal | Manganese sulfate |
| 8 | Corn starch | Corn steep liquor | Calcium carbonate |
| | | powder | |
| 9 | Sucrose | Soybean meal | Calcium carbonate |
| 10 | Sucrose | Corn steep liquor | Manganese sulfate |
| | | powder | |
| 11 | Sucrose | Soybean meal | Magnesium sulfate |
| | | | heptahydrate |
| 12 | Sucrose | Corn steep liquor | Sodium chloride |
| | | powder | |
| 13 | Glucose | Soybean meal | Manganese sulfate |
| 14 | Glucose | Corn steep liquor | Calcium carbonate |
| | | powder | |
| 15 | Glucose | Soybean meal | Sodium chloride |
| 16 | Glucose | Corn steep liquor | Magnesium sulfate |
| | | powder | heptahydrate |
| 17 | Molasses | Corn steep liquor | Magnesium sulfate |
| | | powder | heptahydrate |
| 18 | Molasses | Soybean meal | Sodium chloride |
| 19 | Molasses | Corn steep liquor | Calcium carbonate |
| | | powder | |
| 20 | Molasses | Soybean meal | Manganese sulfate |
| 21 | Corn starch | Corn steep liquor | Sodium chloride |
| | | powder | |
| 22 | Corn starch | Soybean meal | Magnesium sulfate |
| | | | heptahydrate |
| 23 | Corn starch | Corn steep liquor | Manganese sulfate |
| | | powder | |
| 24 | Corn starch | Soybean meal | Calcium carbonate |
| 25 | Sucrose | Corn steep liquor | Calcium carbonate |
| | | powder | |
| 26 | Sucrose | Soybean meal | Manganese sulfate |
| 27 | Sucrose | Corn steep liquor | Magnesium sulfate |
| | | powder | heptahydrate |
| 28 | Sucrose | Soybean meal | Sodium chloride |
| 29 | Glucose | Corn steep liquor | Manganese sulfate |
| | | powder | |
| 30 | Glucose | Soybean meal | Calcium carbonate |
| 31 | Glucose | Corn steep liquor | Sodium chloride |
| | | powder | |
| 32 | Glucose | Soybean meal | Magnesium sulfate |
| | | | heptahydrate |

4 Orthogonal Test Optimization

After finding out the best carbon source, nitrogen source and inorganic salt by single factor test, orthogonal test is designed to further determine the best ratio. The orthogonal test level design is shown in Table 3.

TABLE 2
Orthogonal test factors and levels

Factors

| Level | Nitrogen source | Nitrogen source | Inorganic salt |
|---|---|---|---|
| 1 | 0.3% | 0.5% | 0.5% |
| 2 | 0.5% | 1.0% | 1.0% |
| 3 | 0.7% | 1.5% | 1.5% |

TABLE 3
Orthogonal test design

| Serial number | Nitrogen source | Nitrogen source | Inorganic salt |
|---|---|---|---|
| 1 | 0.3% | 0.5% | 0.5% |
| 2 | 0.3% | 1.0% | 1.5% |
| 3 | 0.3% | 1.5% | 1.0% |
| 4 | 0.5% | 0.5% | 1.5% |
| 5 | 0.5% | 1.0% | 1.0% |
| 6 | 0.5% | 1.5% | 0.5% |
| 7 | 0.7% | 0.5% | 1.0% |
| 8 | 0.7% | 1.0% | 0.5% |
| 9 | 0.7% | 1.5% | 1.5% |

TABLE 4
Viable count of Bacillus velezensis under different
carbon sources, nitrogen sources and inorganic salts

| Serial | Nitrogen | | Inorganic | Viable count |
|---|---|---|---|---|
| number | source | Nitrogen source | salt | (10⁹ cfu/ml) |
| 1 | Molasses | Soybean meal | Magnesium | 1.75 |
| | | | sulfate | |
| | | | heptahydrate | |
| 2 | Molasses | Corn steep liquor | Sodium | 0.45 |
| | | powder | chloride | |
| 3 | Molasses | Soybean meal | Calcium | 0.75 |
| | | | carbonate | |
| 4 | Molasses | Corn steep liquor | Manganese | 1.25 |
| | | powder | sulfate | |
| 5 | Corn | Soybean meal | Sodium | 1.70 |
| | starch | | chloride | |
| 6 | Corn | Corn steep liquor | Magnesium | 1.20 |
| | starch | powder | sulfate | |
| | | | heptahydrate | |
| 7 | Corn | Soybean meal | Manganese | 1.35 |
| | starch | | sulfate | |
| 8 | Corn | Corn steep liquor | Calcium | 1.15 |
| | starch | powder | carbonate | |
| 9 | Sucrose | Soybean meal | Calcium | 0.55 |
| | | | carbonate | |
| 10 | Sucrose | Corn steep liquor | Manganese | 0.45 |
| | | powder | sulfate | |
| 11 | Sucrose | Soybean meal | Magnesium | 1.05 |
| | | | sulfate | |
| | | | heptahydrate | |
| 12 | Sucrose | Corn steep liquor | Sodium | 0.40 |
| | | powder | chloride | |
| 13 | Glucose | Soybean meal | Manganese | 0.55 |
| | | | sulfate | |
| 14 | Glucose | Corn steep liquor | Calcium | 0.60 |
| | | powder | carbonate | |
| 15 | Glucose | Soybean meal | Sodium | 1.65 |
| | | | chloride | |
| 16 | Glucose | Corn steep liquor | Magnesium | 0.75 |
| | | powder | sulfate | |
| | | | heptahydrate | |
| 17 | Molasses | Corn steep liquor | Magnesium | 0.60 |
| | | powder | sulfate | |
| | | | heptahydrate | |
| 18 | Molasses | Soybean meal | Sodium | 1.45 |
| | | | chloride | |
| 19 | Molasses | Corn steep liquor | Calcium | 1.30 |
| | | powder | carbonate | |
| 20 | Molasses | Soybean meal | Manganese | 0.58 |
| | | | sulfate | |
| 21 | Corn | Corn steep liquor | Sodium | 1.30 |
| | starch | powder | chloride | |
| 22 | Corn | Soybean meal | Magnesium | 1.53 |
| | starch | | sulfate | |
| | | | heptahydrate | |
| 23 | Corn | Corn steep liquor | Manganese | 1.25 |
| | starch | powder | sulfate | |
| 24 | Corn | Soybean meal | Calcium | 1.43 |
| | starch | | carbonate | |
| 25 | Sucrose | Corn steep liquor | Calcium | 0.73 |
| | | powder | carbonate | |
| 26 | Sucrose | Soybean meal | Manganese | 0.43 |
| | | | sulfate | |
| 27 | Sucrose | Corn steep liquor | Magnesium | 0.55 |
| | | powder | sulfate | |
| | | | heptahydrate | |
| 28 | Sucrose | Soybean meal | Sodium | 1.03 |
| | | | chloride | |
| 29 | Glucose | Corn steep liquor | Manganese | 0.45 |
| | | powder | sulfate | |
| 30 | Glucose | Soybean meal | Calcium | 0.73 |
| | | | carbonate | |
| 31 | Glucose | Corn steep liquor | Sodium | 1.40 |
| | | powder | chloride | |
| 32 | Glucose | Soybean meal | Magnesium | 1.85 |
| | | | sulfate | |
| | | | heptahydrate | |

TABLE 5
Viable count of bacteria under different ratios of glucose,
soybean meal and magnesium sulfate heptahydrate

| | | | Magnesium | Viable count | |
|---|---|---|---|---|---|
| Serial | | Soybean | sulfate | (10⁹ | |
| number | Glucose | meal | heptahydrate | cfu/ml) | C/N ratio |
| 1 | 0.30% | 0.50% | 0.50% | 1.50 | C/N = 10.2:1 |
| 2 | 0.30% | 1.00% | 1.50% | 1.58 | C/N = 8.5:1 |
| 3 | 0.30% | 1.50% | 1.00% | 1.65 | C/N = 7.9:1 |
| 4 | 0.50% | 0.50% | 1.50% | 1.53 | C/N = 12.5:1 |
| 5 | 0.50% | 1.00% | 1.00% | 1.43 | C/N = 9.6:1 |
| 6 | 0.50% | 1.50% | 0.50% | 1.73 | C/N = 8.7:1 |
| 7 | 0.70% | 0.50% | 1.00% | 1.28 | C/N = 14.8:1 |
| 8 | 0.70% | 1.00% | 0.50% | 2.00 | C/N = 10.8:1 |
| 9 | 0.70% | 1.50% | 1.50% | 1.20 | C/N = 9.4:1 |

At the same time, according to the results, the C/N of the culture medium can be calculated, compare with the C/N (20:1) that is suitable for bacterial fermentation. If the difference is large, the C and N sources are adjusted respectively, to design two groups of culture media with C/N of 20:1, further compare with the best culture media obtained in the earlier stage, so as to select the C/N culture media that is suitable for the growth of Bacillus velezensis. The optimum formula of fermentation medium for Bacillus velezensis XY40-1 is determined by orthogonal test as follows: 0.700% glucose, 1.000% soybean meal, 0.500% magnesium sulfate heptahydrate.

5. Supplement and Optimization of Trace Elements

The common trace elements are iron, copper, manganese, magnesium and zinc. One or more trace element ions selected from cobalt, nickel, molybdenum, selenium and boron can also be added. It is added to the culture medium in the form of chloride or sulfate. The selection of chloride/sulfate refers to the results of acid-base regulator in 6.1. The addition amount of trace elements is 30, 40, 50, 60 μmol/L, screening according to the results, the appropriate trace elements in 2-3 and the added amount are obtained. Further, the composition of trace elements is optimized to form the best trace element addition solution. The highest cell concentration is found when adding 50 μmol/L of zinc sulfate heptahydrate to the medium.

TABLE 6
Viable count of Bacillus velezensis under different trace elements

| Trace element | Viable count (10⁹ CFU/mL) |
|---|---|
| Cobalt chloride | 1.85 |
| Sodium molybdate | 1.95 |
| Boric acid | 1.68 |
| Ferrous sulfate | 1.95 |
| Copper sulphate | 1.83 |
| Zinc sulfate | 2.05 |

6 Optimization of Other Conditions

6.1 pH Test

Based on the optimized medium (0.700% glucose, 1.000% soybean meal, 0.500% magnesium sulfate heptahydrate, 50 μmol/L zinc sulfate heptahydrate), to select acid and alkali preferred by target bacteria, the acid and alkali involved are HCl, H₂SO₄, HNO₃, NaOH, KOH, and Ca(OH)₂. Controlling the consistency of H⁺/OH⁻ concentration to screen out the appropriate acid/alkali. Taking appropriate acid/alkali as pH regulator, pH 4.0, 4.5, 5.0, 5.5, 6.0, 6.5, 7.0, 7.5, 8.0 are respectively selected as the initial value of the culture medium. The bacterial solution with 0.5% of the inoculation amount is inoculated into a 50 mL triangular flask (specification 100 mL) with pH regulated, and the number of viable bacteria is measured after incubating in a shaking table at 30° C. and 160 rpm for 24 h. The results are shown in Table 7. The number of viable bacteria in the culture medium is high at pH 6.5, 7.0, and 7.5. On the basis of the pH value of the preliminary results in Table 7, the pH test is further refined, and the pH is gradually determined to the tenth level. The design pH gradients are 6.5, 6.7, 6.9, 7.1, 7.3 and 7.5. The results are shown in Table 8. When pH=7.3, the number of viable bacteria reaches 2.20×10⁹CFU/mL (the results are shown in Table 8).

TABLE 7
Effect of culture medium with pH in the range of 4.0-8.0
on the viable count of bacteria of Bacillus velezensis

| pH | Viable count (10⁹ CFU/mL) |
|---|---|
| 4.0 | 0.80 |
| 4.5 | 1.10 |
| 5.0 | 1.15 |
| 5.5 | 1.33 |
| 6.0 | 1.48 |
| 6.5 | 1.85 |
| 7.0 | 2.08 |
| 7.5 | 1.78 |
| 8.0 | 1.25 |

TABLE 8
Effect of culture medium with pH in the range of 6.5-7.5
on the viable count of bacteria of Bacillus velezensis

| pH | Viable count (10⁹ CFU/mL) |
|---|---|
| 6.5 | 1.80 |
| 6.7 | 1.75 |
| 6.9 | 1.80 |
| 7.1 | 1.75 |
| 7.3 | 2.20 |
| 7.5 | 1.80 |

6.2 Temperature Test

On the basis of the culture medium in 6.1, selecting 16, 23, 30, 37 and 44° C. as the shaking table temperature, the bacterial solution is inoculated into a 50 mL triangular flask (specification: 100 mL) with 0.5% inoculation amount, pH=7.3, and the number of viable bacteria is measured after incubating in a shaking table at 30° C. and 160 rpm for 24 h. The results are shown in Table 9. The number of viable bacteria at 30° C. and 37° C. is higher than that at 23° C. and 44° C. In order to obtain the optimal temperature, on the temperature value of the preliminary results, the temperature test is further refined, and the temperature is gradually determined to the single-digit level. The design temperature of the shaking table is 31, 33, 35, 37, 39, 41° C. The cell concentration at 30° C. and 37° C. is higher than that at 16° C., 23° C. and 44° C., of which 37° C. is the best, reaching 2.55×10⁹CFU/mL (the results are shown in Table 9-10).

TABLE 9
Effect of Different Temperature of Shaking Table
on the Viable count of Bacillus velezensis

| Temperature (° C.) | Viable count (10⁹ CFU/mL) |
|---|---|
| 23 | 1.45 |
| 30 | 1.98 |
| 37 | 2.45 |
| 44 | 1.55 |

TABLE 10
Effect of Different Temperature of Shaking Table
on the Viable count of Bacillus velezensis

| Temperature (° C.) | Viable count (10⁹ CFU/mL) |
|---|---|
| 31 | 1.93 |
| 33 | 2.13 |
| 35 | 2.25 |
| 37 | 2.55 |
| 39 | 2.45 |
| 41 | 1.95 |

6.3 Rotation Rate Test

On the basis of the culture medium in 6.2, selecting 160, 180, 200, 220, 240 and 260 rpm as the rotation rate of the shaking table, the bacterial solution is inoculated into a 50 mL triangular flask (specification: 100 mL) with 0.5% of the inoculation amount, pH=7.3, and the number of viable bacteria is measured after incubating at 37° C. for 24 hours. Under the rotation rates of 200, 220, and 240 rpm, the bacterial concentration is higher than that of 100, 280 rpm, of which the rotation rate of 220 rpm is the best, and the number of viable bacteria reaches 2.58×10⁹CFU/mL (the results are shown in Table 11)

TABLE 11
Effect of Different Rotation Rate of Shaking Table
on the Viable count of Bacillus velezensis

| Rotation rate (rpm) | Viable count (10⁹ CFU/mL) |
|---|---|
| 160 | 1.61 |
| 180 | 1.73 |
| 200 | 1.87 |
| 220 | 2.58 |
| 240 | 1.94 |
| 260 | 1.82 |

6.4 Fermentation Time Test

On the basis of the culture medium in 6.3, selecting 220 rpm as the rotation rate of the shaking table, the bacterial solution is inoculated into a 50 mL triangular flask (specification: 100 mL) with 0.5% of the inoculation amount, pH=7.3, culturing in a shaking table at 37° C. The culture time of shaking table is set at 18, 20, 22, 24, 26, 28, and 30 h, and then the number of viable bacteria is measured. Under the rotation rate of 22-28 h, the cell concentration is higher than that of 18 h and 30 h. When the fermentation time is 24 h, the viable count of bacteria reaches 2.92×10⁹CFU/mL (the results are shown in Table 12).

TABLE 12
Effects of different fermentation time treatments on the viable
count of bacteria after fermentation by Bacillus velezensis

| Fermentation time (h) | Viable count (10⁹ CFU/mL) |
|---|---|
| 18 | 1.65 |
| 20 | 2.04 |
| 22 | 2.51 |
| 24 | 2.92 |
| 26 | 2.81 |
| 28 | 2.86 |
| 30 | 2.16 |

6.5 Inoculation Amount Test

On the basis of the culture medium in 6.4, selecting 220 rpm as the rotation rate of the shaking table, the bacterial solution is inoculated into a 50 mL triangular flask (specification: 100 mL) at 0.1, 0.3, 0.5, 0.7, 0.9, 1.1, 1.3, 1.5, 1.7% of the inoculation amount, incubating in a shaking table at 37° C. in pH=7.3. After 24 h of shaking table culture, the number of viable bacteria is determined. The results are shown in Table 13. When the inoculation amount is 0.7-1.3%, the number of viable bacteria after fermentation by Bacillus velezensis is 3.06-3.64×10⁹ CFU/mL, when the inoculation amount is 1.1%, the number of viable bacteria after fermentation by Bacillus velezensis can reach 3.64×10⁹CFU/mL (the results are shown in Table 13).

TABLE 13
The effect of treatment on the viable count of bacteria after fermentation
by by Bacillus velezensis under different inoculation amounts

| Inoculation amount (%) | Viable count (10⁹ CFU/mL) |
|---|---|
| 0.1 | 1.65 |
| 0.3 | 2.26 |
| 0.5 | 2.48 |
| 0.7 | 2.69 |
| 0.9 | 3.45 |
| 1.1 | 3.64 |
| 1.3 | 3.06 |
| 1.5 | 2.71 |
| 1.7 | 2.58 |

Based on the above tests, a fermentation method suitable for Bacillus velezensis XY40-1 is obtained, the specific steps are as follows:
Step 1: Preparation of fermentation medium: the formula of fermentation medium is 0.700% glucose, 1.000% soybean meal, 0.500% magnesium sulfate heptahydrate and 0.014% zinc sulfate heptahydrate.
Step 2: Inoculate Bacillus velezensis XY40-1 in the fermentation medium with the inoculation amount of 0.7-1.3% for fermentation. The pH of fermentation medium is 6.5-7.5, the fermentation temperature is 30-39° C., the fermentation speed is 200-240 rpm, and the fermentation time is 20-28 hours. After fermentation, the ultra high performance liquid chromatography-mass spectrometry (LC-MS) will be used for detection (chromatographic column: Acquire UPLC HSS T3 1.8 um 2.1*100 mm), and the fermentation metabolites will contain Nogamycin, Rifaximin, Selamectin, Baicalin and Anthromycin P-3 through detection and analysis.

2, Control Effect of Bacillus velezensis XY40-1 and its Fermentation Products on Plant Diseases

(1) Control Effect of Bacillus velezensis XY40-1 on Plant Diseases

The method of plate confrontation is used: Phytophthora, Geotrichum citrinum, Neopestalotiopsis formicarum and Curvularia are respectively spot connected to the center of PDA plate, and Bacillus velezensis XY40-1 and its fermentation metabolite are spot connected to 2 cm away from the center of the culture medium by cross method. The control group is not inoculated with Bacillus velezensis XY40-1, and cultured at 28° C. for 5-7 days at constant temperature. After Phytophthora, Geotrichum citrinum, Neopestalotiopsis formicarum and Curvularia are grew on the plate, the width of the inhibition zone is measured. Each group is treated for three times.

Inhibition rate (%)=(diameter of control colony−diameter of treated colony)/diameter of control colony*100%

TABLE 14
Inhibitory effect of Bacillus velezensis
XY40-1 on Phytophthora capsici

| | contrast | treatment | |
|---|---|---|---|
| | Diameter of | Diameter of | Inhibition rate of |
| Plant pathogen | pathogen (cm) | pathogen (cm) | pathogen diameter (%) |
| Phytophthora | 8.1 | 1.83 ± 0.15 | 77.47 |
| capsici | | | |

TABLE 15
Inhibition Effect of Bacillus velezensis XY40-1 on Geotrichum candidum,
Neopestalotiopsis formicarum and Curvularia

| | contrast | treatment | |
|---|---|---|---|
| | Diameter of | Diameter of | Inhibition rate of |
| Plant pathogen | pathogen (cm) | pathogen (cm) | pathogen diameter (%) |
| Geotrichum candidum | 7.5 | 3.1 ± 0.39 | 59.00 |
| Neopestalotiopsis | 7.7 | 2.5 ± 0.50 | 68.18 |
| formicarum | | | |
| Curvularia | 6.6 | 2.7 ± 0.52 | 59.85 |

(2) Control Effect of Fermentation Products of Bacillus velezensis XY40-1 on Plant Diseases

Norgamycin (CAS: 1404-15-5), rifaximin (CAS: 80621-81-4), selamectin (CAS: 165108-07-6), baicalin (CAS: 21967-41-9), and anthromycin P-3 (CAS: 66584-72-3) are compounded according to 0.1 mg/ml respectively, and then are respectively inoculated to the PDA medium. After 5 days of culture, Phytophthora capsici, Geotrichum candidum, Neopestalotiopsis formicarum and Curvularia are made into bacterital colonies (d=6 mm) with a hole punch, and then inoculated into the medium containing compound agents, placed in a 25±2° C. incubator for 5-7 days, and measured the colony diameter with a cross method. Three parallels groups are set for each experiment, and the experiment is repeated three times to calculate the antibacterial activity. Inhibition rate (%)=(control colony diameter−treated colony diameter)/control colony diameter*100%, the results are shown in Table 16 and Table 18.

TABLE 16
Inhibition Effect of Mixture of Noganomycin, Rifaximin, Selamectin,
Baicalin and Antinomycin P-3 on Phytophthora capsici

| | contrast | treatment | |
|---|---|---|---|
| | Diameter of | Diameter of | Inhibition rate of |
| Plant pathogen | pathogen (cm) | pathogen (cm) | pathogen diameter (%) |
| Phytophthora | 8.2 | 1.51 ± 0.12 | 81.59 |
| capsici | | | |

TABLE 17
Inhibitory Effect of Mixture of Noganomycin, Rifaximin, Selamectin,
Baicalin and Amphotericin P-3 on Geotrichum candidum,
Neopestalotiopsis formicarum and Curvularia

| | contrast | treatment | |
|---|---|---|---|
| | Diameter of | Diameter of | Inhibition rate of |
| Plant pathogen | pathogen (cm) | pathogen (cm) | pathogen diameter (%) |
| Geotrichum candidum | 7.4 | 1.1 ± 0.32 | 85.14 |
| Neopestalotiopsis | 7.8 | 1.0 ± 0.45 | 87.18 |
| formicarum | | | |
| Curvularia | 6.5 | 1.3 ± 0.48 | 80.00 |

The mixture is obtained by mixing 0.1 mg/ml of norganomycin (CAS: 1404-15-5), rifaximin (CAS: 80621-81-4), selamectin (CAS: 165108-07-6), baicalin (CAS: 21967-41-9) and angiosporin P-3 (CAS: 66584-72-3). The root and leaf of pepper are inoculated with phytophthora capsici after being scratched, and are divided into experimental group and control group. The root and leaf of citrus are inoculated with Geotrichum candidum after being scratched, and are divided into experimental group and control group. The roots and leaves of coconuts are inoculated with Neopestalotiopsis formicarum after being scratched, and are divided into experimental group and control group. The root and leaf of corn are inoculated with Curvularia after being scratched, and are divided into experimental group and control group. The compound mixture are sprayed on the roots and leaves of the plants in the experimental group, to observe the incidence of the disease in the experimental group and the control group, and the disease inhibition rate are calculated after 25 days later, as shown in Table 18:

TABLE 18
Inhibition Effect of Mixture of Noganomycin, Rifaximin, Selamectin,
Baicalin and Anthromycin P-3 on Plant Diseases

| | Mixture | | | |
|---|---|---|---|---|
| | 0.1 mg/ml Noganomycin + 0.1 mg/ml Rifaximin + 0.1 mg/ml | | | |
| | Selamectin + 0.1 mg/ml Baicalin + 0.1 mg/ml Anthromycin P-3 | | | |
| | Phytophthora | Citrus acid rot | Neopestalotiopsis | |
| | capsici | (Geotrichum | formicarum | Corn Leaf Spot |
| Disease type | (Phytophthora) | candidum) | (Pestalotiopsis) | (Curvularia) |
| Disease | 78.6% | 80.2% | 83.1% | 76.4% |
| inhibition rate | | | | |

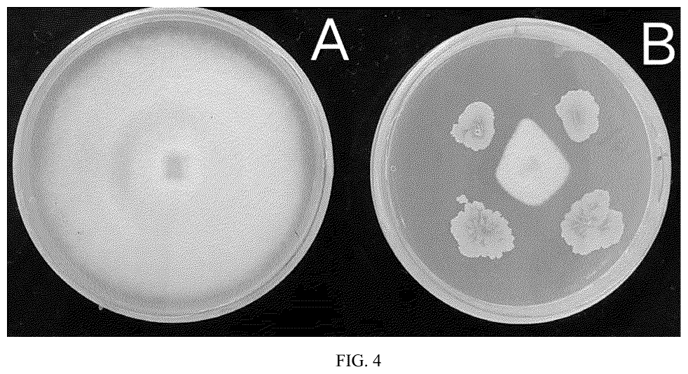

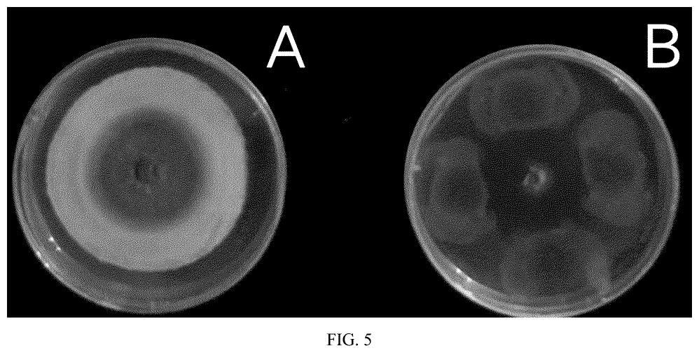

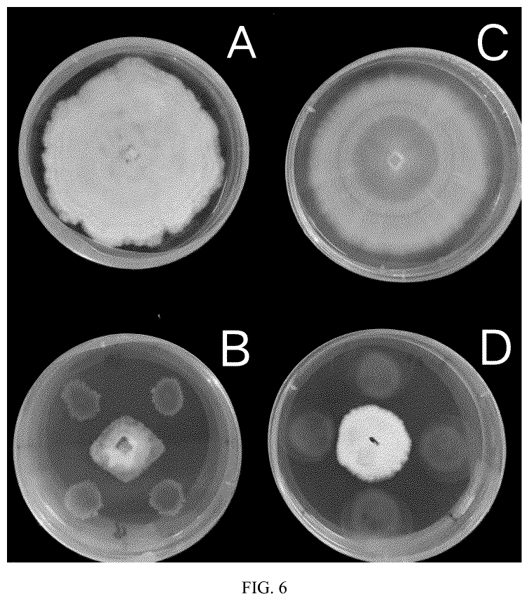

The antagonistic effect of Bacillus velezensis XY40-1 and its fermentation metabolite (containing norgamycin, rifaximin, selamectin, baicalin and amphotericin P-3) against phytophthora pestis, geotrichum candidum, coconut gray spot and curvularia lunata is shown in FIGS. 4-6 and Table 14-18. Through the plate confrontation cultivation method, the Bacillus velezensis XY40-1 and its fermentation metabolite of the disclosure have good antagonism against geotrichum candidum, coconut gray spot and curvularia lunata.

Sequence Listing

```
<?xml version=“1.0” encoding=“UTF-8”?>
<!DOCTYPE ST26SequenceListing PUBLIC “-//WIPO//DTD Sequence Listing 1.3//EN”
“ST26SequenceListing_V1_3.dtd”>
<ST26SequenceListing dtdVersion=“V1_3” fileName=“SEQLISIT.xml” softwareName=“WIPO
Sequence” software Version=“2.2.0” productionDate=“2023-01-10”>
  <ApplicationIdentification>
    <IPOfficeCode></IPOfficeCode>
    <ApplicationNumberText></ApplicationNumberText>
    <FilingDate>2023-01-09</FilingDate>
  </ApplicationIdentification>
  <ApplicantFileReference>ASF1234566</ApplicantFileReference>
  <EarliestPriorityApplicationIdentification>
    <IPOfficeCode>CN</IPOfficeCode>
    <ApplicationNumberText>202210422749.X</ApplicationNumberText>
    <FilingDate>2022-04-21</FilingDate>
  </EarliestPriorityApplicationIdentification>
  <ApplicantName languageCode=“en”>Hunan Vegetable Research Institute</ApplicantName>
  <ApplicantNameLatin>Hunan Vegetable Research Institute</ApplicantNameLatin>
  <InventionTitle languageCode=“en”>BACILLUS VELEZENSIS, FERMENTATION
METHOD, FERMENTATION PRODUCT AND ITS APPLICATION</InventionTitle>
  <SequenceTotalQuantity>3</SequenceTotalQuantity>
  <SequenceData sequenceIDNumber=“1”>
    <INSDSeq>
      <INSDSeq_length>20</INSDSeq_length>
      <INSDSeq_moltype>DNA</INSDSeq_moltype>
      <INSDSeq_division>PAT</INSDSeq_division>
      <INSDSeq_feature-table>
        <INSDFeature>
          <INSDFeature_key>source</INSDFeature_key>
        <INSDFeature_location>1 . . . 20</INSDFeature_location>
        <INSDFeature_quals>
          <INSDQualifier>
            <INSDQualifier_name>mol_type</INSDQualifier_name>
            <INSDQualifier_value>other DNA</INSDQualifier_value>
          </INSDQualifier>
          <INSDQualifier id=“q2”>
            <INSDQualifier_name>organism</INSDQualifier_name>
            <INSDQualifier_value>synthetic construct</INSDQualifier_value>
          </INSDQualifier>
        </INSDFeature_quals>
      </INSDFeature>
    </INSDSeq_feature-table>
    <INSDSeq_sequence>agagtttgatcctggctcag</INSDSeq_sequence>
  </INSDSeq>
</SequenceData>
<SequenceData sequenceIDNumber=“2”>
  <INSDSeq>
    <INSDSeq_length>21</INSDSeq_length>
    <INSDSeq_moltype>DNA</INSDSeq_moltype>
    <INSDSeq_division>PAT</INSDSeq_division>
    <INSDSeq_feature-table>
      <INSDFeature>
        <INSDFeature_key>source</INSDFeature_key>
        <INSDFeature_location>1 . . . 21</INSDFeature_location>
        <INSDFeature_quals>
          <INSDQualifier>
            <INSDQualifier_name>mol_type</INSDQualifier_name>
            <INSDQualifier_value>other DNA</INSDQualifier_value>
          </INSDQualifier>
          <INSDQualifier id=“q4”>
            <INSDQualifier_name>organism</INSDQualifier_name>
            <INSDQualifier_value>synthetic construct</INSDQualifier_value>
          </INSDQualifier>
        </INSDFeature_quals>
      </INSDFeature>
    </INSDSeq_feature-table>
    <INSDSeq_sequence>tacggctaccttgttacgact</INSDSeq_sequence>
  </INSDSeq>
</SequenceData>
<SequenceData sequenceIDNumber=“3”>
  <INSDSeq>
    <INSDSeq_length>1449</INSDSeq_length>
    <INSDSeq_moltype>DNA</INSDSeq_moltype>
    <INSDSeq_division>PAT</INSDSeq_division>
    <INSDSeq_feature-table>
      <INSDFeature>
        <INSDFeature_key>source</INSDFeature_key>
        <INSDFeature_location>1 . . . 1449</INSDFeature_location>
        <INSDFeature_quals>
          <INSDQualifier>
            <INSDQualifier_name>mol_type</INSDQualifier_name>
            <INSDQualifier_value>genomic DNA</INSDQualifier_value>
          </INSDQualifier>
          <INSDQualifier id=“q6”>
            <INSDQualifier_name>organism</INSDQualifier_name>
            <INSDQualifier_value>Bacillus velezensis</INSDQualifier_value>
          </INSDQualifier>
        </INSDFeature_quals>
      </INSDFeature>
    </INSDSeq_feature-table>
  <INSDSeq_sequence>ggagcttgctccctgatgttagcggcggacgggtgagtaacacgtgggtaacctgcctgtaagactgg
gataactccgggaaaccggggctaataccggatggttgtctgaaccgcatggttcagacataaaaggtggcttcggctaccacttacagatgga
cccgcggcgcattagctagttggtgaggtaacggctcaccaaggcgacgatgcgtagccgacctgagagggtgatcggccacactgggact
gagacacggcccagactcctacgggaggcagcagtagggaatcttccgcaatggacgaaagtctgacggagcaacgccgcgtgagtgatga
aggttttcggatcgtaaagctctgttgttagggaagaacaagtgccgttcaaatagggcggcaccttgacggtacctaaccagaaagccacggc
taactacgtgccagcagccgcggtaatacgtaggtggcaagcgttgtccggaattattgggcgtaaagggctcgcaggcggtttcttaagtctga
tgtgaaagcccccggctcaaccggggagggtcattggaaactggggaacttgagtgcagaagaggagagtggaattccacgtgtagcggtga
aatgcgtagagatgtggaggaacaccagtggcgaaggcgactctctggtctgtaactgacgctgaggagcgaaagcgtggggagcgaacag
gattagataccctggtagtccacgccgtaaacgatgagtgctaagtgttagggggtttccgccccttagtgctgcagctaacgcattaagcactcc
gcctggggagtacggtcgcaagactgaaactcaaaggaattgacgggggcccgcacaagcggtggagcatgtggtttaattcgaagcaacgc
gaagaaccttaccaggtcttgacatcctctgacaatcctagagataggacgtccccttcgggggcagagtgacaggtggtgcatggttgtcgtca
gctcgtgtcgtgagatgttgggttaagtcccgcaacgagcgcaacccttgatcttagttgccagcattcagttgggcactctaaggtgactgccgg
tgacaaaccggaggaaggtggggatgacgtcaaatcatcatgccccttatgacctgggctacacacgtgctacaatggacagaacaaagggc
agcgaaaccgcgaggttaagccaatcccacaaatctgttctcagttcggatcgcagtctgcaactcgactgcgtgaagctggaatcgctagtaat
cgcggatcagcatgccgcggtgaatacgttcccgggccttgtacacaccgcccgtcacaccacgagagtttgtaacacccgaagtcggtgagg
taacctttatggagccagccgccgaaggtgggacagatgattggggtgaagtcgtaacaaggtagccgtatcgg</INSDSeq_sequence>
      </INSDSeq>
  </SequenceData>
    </ST26SequenceListing>
```

What is claimed is:

1. A strain of Bacillus velezensis XY40-1, which is characterized in that the Bacillus velezensis XY40-1 was stored in China Center for Type Culture Collection on Mar. 29, 2022, with the deposit number of CCTCC NO: M 2022342.

2. A microbial inoculum, wherein the microbial inoculum comprises Bacillus velezensis XY40-1 according to claim 1.

3. The microbial inoculum according to claim 2, wherein the microbial inoculum is a liquid microbial inoculum or a solid microbial inoculum.

4. An application of the Bacillus velezensis XY40-1 according to claim 1, wherein to inhibit a growth of plant pathogens.

5. The application according to claim 4, wherein the plant pathogen comprises any one of Geotrichum candidum, Curvularia and Neopestalotiopsis formicarum.

6. A fermentation method of Bacillus velezensis XY40-1 according to claim 1, which is characterized in that the method comprises the following steps:
(1) preparing of fermentation medium: a formula of the fermentation medium is 0.700% glucose, 1.000% soybean meal, 0.500% magnesium sulfate heptahydrate and 0.014% zinc sulfate heptahydrate; pH is 6.5-7.5;
(2) inoculating Bacillus velezensis XY40-1 with an inoculation amount of 0.7-1.3% into the fermentation medium for fermentation; a fermentation temperature is 30-39° C.; a fermentation speed is 200-240rpm; a fermentation time is 22-28 hours.

7. The fermentation method according to claim 6, wherein the pH in step (1) is 7.3; in step (2), the inoculation amount is 1.1%, the fermentation temperature is 37° C., the fermentation speed is 220 rpm, and the fermentation time is 24 hours.

8. An application of a fermentation product obtained by the fermentation method according to claim 6 in inhibiting the growth of plant pathogens.

9. The application according to claim 8, wherein the plant pathogen comprises any one of Geotrichum candidum, Curvularia and Neopestalotiopsis formarum.

10. The application according to claim 8, wherein the fermentation product contains nogalamycin, rifaximin, selamectin, baicalin and ansamitocin P-3.

11. An application of the Bacillus velezensis XY40-1 according to any microbial inoculum according to claims 2 to inhibit a growth of plant pathogens.

12. The application according to claim 11, wherein the plant pathogen comprises any one of Geotrichum candidum, Curvularia and Neopestalotiopsis formicarum.
